(19) United States
(12) Patent Application Publication
(10) Pub. No.: US 2016/0030495 A1
(43) Pub. Date: Feb. 4, 2016

(54) METHODS AND COMPOSITIONS FOR BACTERIOPHAGE THERAPY

(71) Applicant: THE BOARD OF REGENTS OF THE UNIVERSITY OF TEXAS SYSTEM, Austin, TX (US)

(72) Inventor: Philip SERWER, San Antonio, TX (US)

(21) Appl. No.: 14/776,094

(22) PCT Filed: Mar. 14, 2014
(86) PCT No.: PCT/US14/27099

§ 371 Date: Sep. 14, 2015

Related U.S. Application Data
(60) Provisional application No. 61/800,684, filed on Mar. 15, 2013.

(51) Int. Cl. A61K 35/76; C12N 7/00

(52) U.S. Cl. CPC A61K 35/76; C12N 7/00; C12N 2795/00032; C12N 2795/00021

(57) ABSTRACT

Embodiments are directed to methods and composition for preparing and using therapeutic phage.

This Application claims priority to U.S. Provisional Application Ser. No. 61/800,684 filed Mar. 15, 2014, which is incorporated herein by reference in its entirety.

### STATEMENT REGARDING FEDERALLY FUNDED RESEARCH

This invention was made with government support under GM24365 awarded by the National Institutes of Health. The government has certain rights in the invention.

### BACKGROUND

Embodiments of this invention are directed generally to microbiology and medicine. Certain aspects are directed to both therapeutic compositions comprising phage and methods of using the same.

### SUMMARY

An advantage of phage therapy is that phages replicate while they are exerting their therapeutic effect. As a result, the number of phages (dose size) used for treatment of infection can be relatively small, and phage therapy can propagate among animals. Another advantage is the specificity of phage for the bacteria targeted for removal. Typically, most other bacteria are not killed. In addition, phage therapy can be rapidly modified, because isolating new phages usually takes only 1-2 weeks. Also, phage therapy has no known side effects (Inal, Arch Immunol Ther Exp (2003), 51:237-244).

The inventors have developed efficient methods for the rapid isolation of improved therapeutic phages. Certain embodiments are directed to combinations of these methods in order to dramatically improve the effectiveness of phage therapy.

Certain embodiments are directed to methods of treating a pathogenic bacteria infection comprising: (a) isolating two or more bacteriophage that lyse the pathogenic bacteria by (i) placing an environmental sample on the surface of a solid nutrient broth growth medium; (ii) applying ultra dilute (0.05%-0.02%) molten agarose containing the pathogenic bacteria and a nutrient broth onto the surface of the solid growth medium to completely or partially cover the environmental sample; (iii) incubating agarose covered growth medium the pathogenic bacteria form a bacterial lawn in the ultra-dilute agarose layer and bacteriophage in the environmental sample form plaques in the pathogenic bacterial lawn; (iv) sampling a plaque present in the ultradilute molten agarose; (v) transferring the bacteriophage in the sampled plaque to a second agar growth medium; (vi) applying a second ultra dilute (0.05%-0.02%) molten agarose containing the pathogenic bacteria and a nutrient broth to the second solid growth medium; (ix) incubating the ultradilute agarose and second agar growth medium until the pathogenic bacteria formed a bacterial lawn in the ultra-dilute agarose layer and the transferred bacteriophage form plaques in the pathogenic bacterial lawn; and (x) isolating the bacteriophage from the plaques from the second ultradilute agaraose; and (b) formulating and administering two or more isolated bacteriophage that target the pathogenic bacteria infecting the subject. In certain aspects the bacteriophage are long-genome bacteriophage. In a further aspect the long-genome bacteriophage are administered in combination with other bacteriophage.

In certain aspects a phage therapy is implemented with a mixture of phages (cocktail). The phages in such a composition can be optimized for the bacteria targeted. The use of a cocktail maximizes the range of bacterial targets and minimizes the emergence of bacterial resistance.

Certain embodiments are directed to methods of isolating 1, 2, 3, 4, or more therapeutic phage. The methods include one or more of the following: (a) placing an environmental sample on the surface of a solid nutrient broth growth medium; (b) applying molten agarose containing host bacteria cells (e.g., a target pathogenic bacteria) (in certain embodiments the agarose is ultra dilute (0.05%-0.02%) agarose) and nutrient broth onto the surface of the solid growth medium to completely or partially cover the environmental sample; (c) allowing the molten agarose to gel; (d) incubating the culture until the bacteria formed a bacterial lawn in the agarose layer and bacteriophage form zones of lawn-clearing around a portion of the environmental sample; (e) sampling a plaque (e.g., inserting a platinum wire into the plaque present in the agarose to the agar of a second solid nutrient broth growth medium; (f) inoculating a second growth medium (e.g., stabbing the needle into the agar of a second plate); (g) applying molten agarose (e.g., ultra dilute (0.05%-0.02%) containing host bacteria cells (e.g., target pathogenic bacteria) and nutrient broth onto the surface of the second solid growth medium; (h) rocking the dish to partially spread the phages; (i) allowing the molten agarose to gel and incubating the culture until the bacteria formed a bacterial lawn in the ultra-dilute agarose layer and bacteriophage form zones of lawn-clearing; and (j) coring plaques.

The methods can further comprise placing plaque cores in a cryo-protecting solution. The cryo-protected core can be frozen at −70° C. or below. In certain aspects the cryo-protectant is 10% dextran 10,000.

Certain embodiments are directed to a phage composition comprising (a) long genome, lytic phage, and (b) lysis enhancing polymer. In certain aspects the compositions is a dry phage composition comprising at least two long-genome lytic phages that specifically bind and lyse a pathogenic bacterium. In certain aspects the pathogenic bacterium is Salmonella enterica, Listeria monocytogenes, Escherichia coli (O157:H7), Campylobacter jejuni, Staphylococcus aureus, and Clostridium perfringens.

Certain aspects are directed to treating a subject having a bacterial infection comprising administering a long-genome, lytic phage composition comprising at least two phage that bind and lyse a pathogenic bacterium.

The term “effective amount” means an amount effective, at dosages and for periods of time necessary, to achieve the desired therapeutic or prophylactic result.

An “effective amount” of an anti-bacterial agent, in reference to decreasing bacterial cell growth, means an amount capable of decreasing, to some extent, the growth of targeted bacteria. The term includes an amount capable of invoking a growth inhibitory, cytostatic and/or cytotoxic effect and/or killing of targeted bacteria.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION

Infections of farm and ranch animals cause tens of billions of dollars of losses per year, including 5-10 billion dollars from food-borne illness. These infections are primarily caused by six pathogens, Salmonella enterica, Listeria monocytogenes, Escherichia coli (0157:H7), Campylobacter jejuni, Staphylococcus aureus and Clostridium perfringens. (Buzby et al., Bacterial foodborne disease: Medical costs and productivity losses. Economic Research Service/USDA, available via the world wide web at ers.usda.gov/media/755604/aer741a_{—}1_.pdf).

These bacterial infections are most frequently treated by the administration of antibiotics. However, antibiotic treatment is sometimes not effective, especially when the infection is biofilm-based, and in some cases, antibiotics promote the formation of biofilms (Costa et al., Vet Microbiol (2012), 160, 488-90). Furthermore, the continued use of antibiotics over years has led some of these bacteria to become resistant to multiple antibiotics (multi-antibiotic resistant) (Saini et al., J Dairy Sci (2012), 95, 4319-32).

Historically, antibiotic therapy was predated by therapies that employed bacterial viruses (bacteriophages or phages) as agents to kill pathogenic bacteria (Summers, Annu Rev Microbiol (2001), 55:437-51; Huff et al., Avian Diseases (2003), 47:1399-1405; Johnson et al., Animal Health Res Rev (2008), 9:201-15; Clark and March, TRENDS Biotechnol (2006), 24:212-18; Kutter et al., Curr Pharmaceut Biotechnol (2012), 11:69-86). This phage therapy has also been used to treat farm and ranch animals such as chickens, hogs, and cattle (Summers, Annu Rev Microbiol (2001), 55:437-51; Huff et al., Avian Diseases (2003), 47:1399-1405; Johnson et al., Animal Health Res Rev (2008), 9:201-15). In some cases, for example in the treatment of typhoid fever, phage therapy was dramatically effective. However, it was not always effective for treatment of farm/ranch animals or humans (Summers, Annu Rev Microbiol (2001), 55:437-51; Huff et al., Avian Diseases (2003), 47:1399-1405; Johnson et al., Animal Health Res Rev (2008), 9:201-15; Clark and March, TRENDS Biotechnol (2006), 24:212-18; Kutter et al., Curr Pharmaceut Biotechnol (2012), 11:69-86). Most early studies on phage therapy were not rigorously controlled.

Other than some isolated success with the treatment of E. coli-caused diarrhea in calves, advances in phage therapy have been incremental and largely disappointing. Needed are more effective phages, more rapid methods for isolating and recognizing effective phages, and more effective multi-phage-containing cocktails, including cocktails that can be transported and used in a dry, ambient temperature-state. These methods must be combined to improve the phage therapy and prophylaxis of the infectious diseases of both humans and farm and ranch animals.

Certain embodiments are directed to development of antibiotic-enhancing or antibiotic-free methods and/or compositions for managing infections of humans and animals, e.g., farm and ranch animals.

A primary reason for development of a comprehensive combination of various methods and compositions is the recent development of rapid procedures for single-plaque propagating and identifying long-genome (>200 Kb) phages. Many long-genome phage were not previously detectable, are ultra-lytic and have other characteristics that suggest dramatically increased effectiveness for phage therapy, including effectiveness for biofilm-carried bacteria.

Certain embodiments are directed to combining methods for (1) isolating and identifying the most effective phages, including long-genome phages, (2) characterizing genomes and/or phenotypes of long-genome phages; (2) optimizing effects of several factors, including polymers, on phage aggressiveness; and (3) optimizing the phage cocktail formulation, including use of dried, lytic phage cocktails for lysing targeted bacterial hosts and minimizing production of phage-resistant mutants.

I. Methods for Isolating Large Lytic Phage from a Solid Environmental Sample

Methods have been developed to isolate and identify large-genome, lytic phages in order to access phages that had not been identifiable by standard phage isolation methods. Large lytic phages contain a diverse genome that helps these phages adapt to diverse environments, including those to be encountered during phage therapy. But, large phages do not propagate well in traditional agar gels and they might be hard to extract from solid environmental surfaces, especially if in biofilms.

An example of long-genome phage isolation follows. A solid environmental sample of dry soil was placed on the surface of a solid (1% agar) nutrient broth growth medium in a Petri dish. Ultra dilute (0.05%-0.02%) molten agarose containing host bacterial cells and nutrient broth was poured onto the surface of the solid growth medium to completely or partially cover the environmental sample. The agarose solution was allowed to gel; care was taken not to disturb the cultures during incubation, and the Petri dish was incubated until the bacteria formed a turbid “lawn” in the ultra-dilute agarose layer. Bacteriophages in the solid environmental sample diffused into the ultra-dilute agarose layer and formed zones of lawn-clearing around the environmental particle that served as source of the phages. The zones of clearing resulted from the phage-induced lysis (bursting) of bacterial cells of the lawn in the ultra-dilute agarose.

For purifying phages from the zones of clearing, a platinum needle was used to transfer the phages to the agar of a second solid (1% agar) nutrient broth growth medium in a Petri dish. The needle was stabbed into a zone of clearing and then stabbed into the agar of the second Petri dish. Ultra dilute (0.05%-0.02%) molten agarose containing host bacteria cells and nutrient broth was poured onto the surface of the solid growth medium having the newly transferred phages and the dish was mildly rocked to partially spread the phages. After incubation and formation of a turbid bacterial lawn, the result was (1) a cleared zone, centered on the stabs, that was the product of serial lysis of cells by descendants of several phages of the original, stabbed inoculum and (2) circular zones of clearing (plaques) that were likely the product of serial lysis of cells by descendants of a single phage of the original inoculum. The cloning procedure can be repeated 1, 2, 3, 4 or more times. Four plaques of cloned phage were cored from a final Petri dish and dropped into a cryoprotecting solution in a small conical tube and frozen at −70° C. The cryo-protectant was 10% dextran 10,000.

The procedure described above is simpler and less costly than traditional phage isolation and has several advantages. (1) The use of ultralow concentration agarose gels for the propagation of environmental phages not detectable with other procedures, especially large and aggregating, highly lytic phages (Serwer et al. Virology (2004), 329:412-24; Thomas et al., Virology (2008), 376:330-38; Serwer et al., Virol J (2007), 4:21). (2) The capacity for use of in situ fluorescence microscopy for rapid initial characterization of newly isolated phages and their genomes (Serwer et al. J Microsc (2007), 228:309-21). (3) The capacity for rapid DNA isolation (without purification of phages) for use in sequencing and informatics analysis, such as high-throughput pyrophosphate-based sequencing (pyrosequencing). (4) The capacity for use of analytical ultracentrifugation with fluorescence detection to determine phage aggregation (Serwer et al., Methods Mol Biol (2009), 501:55-66).

### II. Promotion of Lytic Character of Large Genome Phages

Some genomically and phenotypically lytic phages (including B. thuringiensis phage, 0305φ8-36) do not visibly lyse liquid cultures, although they do produce clear plaques when plated in ultra-dilute agarose overlays (Serwer et al., Methods Mol Biol (2009), 501:55-66; Serwer et al., Virol J (2007), 4:21). The primary differences between the conditions of liquid culture and the conditions of plaque formation are, first, the level of aeration and, second, the presence of ultra-dilute agarose during plaque formation, but not during liquid culture. So, to determine whether the presence/absence of agarose was the cause of the difference in cell lysis, the concentration of agarose was progressively reduced in the upper layer during plating, and plated with phage 0305φ8-36 in a concentration sufficient to produce confluent lysis in the presence of 0.15% agarose. The upper layer agarose solution formed a weak gel for 0.05% and more concentrated agarose, but remained liquid for 0.04% and more dilute agarose. Thus, care was taken not to disturb the cultures during incubation. After incubation, it was found that presence of either 0.02% or more concentrated agarose resulted in a clear culture; presence of 0.01% or more dilute agarose resulted in a turbid culture (unpublished data). In other words, the presence of 0.02% or more concentrated agarose had been essential to the lytic phenotype that assisted initial detecting of this phage.

The phage isolation rate with our dilute gel- and needle transfer-based procedure is far above what is normally achieved (1-2 orders of magnitude above). The more phages that are isolated and available for cocktails, the more optimal will be the cocktail for phage therapy.

The phages isolated are of widely varying type and are usually lytic. Some are either extreme versions of what is already known and others are phages of completely new type. In certain instances phages were sequenced. The sequence of one of the larger phages, 201φ2-1 (genome length=316.674 Kb) had an entire region enriched with genes for which no homologs exist in either smaller phages or anywhere else. Yet, these genes are functional in the natural environment because no sequence-based evidence exists that they are either degraded or degrading (Thomas et al., Virology (2008), 376:330-38).

Thus, the assumption is that a function of these genes of unknown function is to adapt the phage to propagation in the various niches that it will encounter in the wild. This point is important for phage therapy because phage therapy occurs in a complex environment, not on a Petri plate where the phages for phage therapy are propagated. Thus, (a) many or most of these genes of unknown function will contribute significantly to the effectiveness of phage therapy and (b) optimization of phage therapy includes minimizing the loss or damage of the genes of unknown function. Nonetheless, we do not have to know the function of these genes in order to use them to achieve improved phage therapy.

Long genome phage often cannot be isolated by any means other than the ultra-dilute-agarose gel-based procedure described above. The reasons for this are: (i) The pores of the agar gels traditionally used for phage plating are small enough so that some large phages do not have enough clearance to propagate. (ii) In addition, some large phages systematically aggregate during propagation, which adds to their effective size in inhibiting propagation in traditional agar gels. (iii) Either aggregation or another quorum-sensing device activates as some phages increase in amount, thereby inhibiting phage propagation before all hosts are killed. This characteristic was presumably selected to optimize long-term phage propagation and prevents visible lysis of liquid cultures. Thus, these phages are undetectable by liquid enrichment culture. The dilute agarose present during the procedure can actually stimulate phage propagation beyond what it is in liquid culture. This latter observation suggests the use of either ultra-dilute agarose or possibly a more effective polymer as adjuvant for phage propagation during phage therapy.

Finally, the aggregation characteristic of at least one large phage is lost during laboratory propagation (6× single plaque propagation_{36?}), which suggests that aggregation is not of selective value in the laboratory and that it is of selective value in the wild; only one aggregating phage has been tested in this way, thus far. This observational knowledge has suggested the following hypothesis. Some aggregating phages have a life cycle that includes a phage's becoming a structural part of biofilms formed by the host. Such endogenous biofilm phages might be “activatable” for “endogenous phage therapy” of biofilm-dependent infections, including bovine mastitis.

By omitting liquid culture after single-plaque purification the number of serial infections are minimized before both storage and cocktail production. This aspect is useful to avoid the loss or damage of genes that are not advantageous in the laboratory, but do provide a competitive advantage for the phage in the wild. Previous conditions in the wild are presumably more similar than laboratory conditions to conditions during phage therapy.

Soil samples used for isolation of environmental phages were dry and most had, just before removal (i.e., within <24 hours), experienced temperatures in the 49-60° C. range. Therefore, dry conditions exist for the storing and transporting these phages at ambient temperature. Storage and transport at ambient temperature, when achieved, will be a major advantage for next-generation phage cocktails.

Pre-cocktail procedures minimize potential for formation of aerosols in that they do not involve liquid culture. Thus, safety is increased and the probability of phage contaminations is decreased.

The inventor has developed the following three-part observational knowledge-based hypothesis relevant to the development next-generation, highly effective phage therapy. (1) The effectiveness of phage therapy, especially for biofilm-propagated infections, will be dramatically increased if the current collection of phages is de-emphasized and use of lytic phages of new types, including long-genome phages, is adopted. (2) The effectiveness of some of these phages will be optimized by the inclusion of polymers in the cocktails used to manage bacterial infections. (3) To further optimize procedures, stably infective cocktails of phages should (and can) be developed with much better characteristics than in the past. These characteristics include dry, ambient-temperature storage, transport and delivery.

Certain aspects are directed to rapid (30-90 minutes) in situ (in-plaque) fluorescence microscopy to determine the extent to which phage particles are aggregated. Extensive aggregation was observed for some phages.

Certain aspects use plaque diameter vs. upper layer agarose gel concentration to obtain a rough idea of how large a phage is, even before single plaque purification had been completed. Ultra-small, extremely clear, sharp-boundary plaques in a 0.2% agarose overlay were found to be a very good indication of a large (genome length >200 Kb), lytic phage (>80% correlation).

Certain aspects use procedures for purifying sequencing quality phage genomic DNA from crude plate lysates without purifying phage particles (Serwer et al., Electrophoresis (2007), 28:1896-902). The procedure involves digesting all DNA that was not phage DNA and then phenol extracting the phage DNA. Just before phenol extraction, the DNA was in excellent condition for analysis of both DNA length and DNA conformation (linear, circular) by use of pulsed field gel electrophoresis (PFGE). The PFGE was performed in either field inversion mode or our in-house developed rotating gel mode.

Certain aspects are directed to procedures for the ultracentrifugal purifying of phages that were unstable in the cesium chloride or other density gradients usually used for ultracentrifugal phage purification (Pathria et al., Bacteriophage (2012), 2:25-35). Over half of large environmental phages are unstable in cesium chloride density gradients.

With these procedures, large, lytic phages can be rapidly isolated and rapidly (days) identified and characterized at a rate much (˜100×) higher than the rate of subsequent sequencing-based genome analysis. Sequence-based confirmation that the phages to be used were genomically lytic, i.e., the phages did not have genes for lysogeny, would assist in characterizing phage as therapeutic phage. The reason is that lysogeny is associated with induction of bacterial pathogenesis, in part via phage-encoded toxins, such as cholera, diphtheria, botulinum and erythrogenic toxins.

Our newly proposed combination of methods described herein (originally developed for phage genomics, not phage therapy) will dramatically enhance the success of both phage therapy and phage prophylaxis by utilizing recently developed methods and recently obtained data to (1) isolate lytic phages and characterize (including genomic sequencing/annotation) them at a rate much higher than in the past, (2) isolate new lytic phages that were not accessible to isolation in the past and that have characteristics that project higher effectiveness in phage therapy and prophylaxis, including effectiveness for bacteria in biofilms, (3) the use of polymer additives for stimulating phage propagation during phage therapy and prophylaxis, and (4) improve the storage, retrieval and transportation of phages and phage cocktails so that they can be transported dry at ambient temperature.

Certain methods begin with the choosing of the initial bacterial target hosts for isolating phages, such as Salmonella enterica, serotype typhimurium (Salmonella typhimurium), and E. coli (e.g., 0157:H7).

A second step will be to choose soil samples from which to isolate phages. The focus is on dry samples because of past success and also because the phages isolated are expected to be amenable to being stored and shipped dry, at ambient temperature. These methods are used to obtain a collection of highly diverse, lytic phages from which optimal therapeutic phage(s) are selected.

During the initial formation of soil particle-derived cleared zones in a bacterial lawn, the bacteria making the lawn have at least two mechanisms for suppressing non-self bacteria from soil particles, (a) swarming and (b) secreting a diffusible compound that is non-toxic to the secreting bacteria, but toxic to most others.

We anticipate that the rate and range of phage isolations can be increased by varying conditions of propagation of host and/or phage. These conditions include temperature of propagation and pH of the medium. In addition, the types and amounts of additives used to promote the initial propagation of lytic phages can be optimized, while introducing bias toward large phages that are adapted to infecting biofilm-forming bacteria.

The phage genomes are partially or completely sequenced. The sequences are scanned for genes associated with lysogeny. Phages with these genes are excluded. Phages that pass this screen proceed to further annotation.

Lytic phage aggressiveness is likely to depend on the composition of polymers present. Given the prospect that these polymers will eventually be used in contact with the tissues of either animals or humans, the polymers are selected from those polymers known to be compatible with application to humans. These compounds include agarose of various types, which sometimes is chemically derivatized with either methyl or ethyl groups. Agarose is a subfraction of agar. Agar is used in food. Other polymers include, but are not limited to dextran, curdlan, and xanthan gum, which are human tissue compatible. Additional polymers can include those used for rheology in cosmetics and implantable medical devices, including catheters. These include mixtures that contain xanthan gum, polyethylene glycols, and polyacrylates among other compounds. The reason is that the natural habitat of the phage is usually very dry and polymer-rich. Phage reproduction can only begin when rain arrives. For dry, polymer-embedded phages, the first consequence of rain is hydrating of the phage-embedding polymer. Thus, by this reasoning, phages would be selected for responding to hydrated polymer by entering an aggressive state, possibly by changing the position of tail associated proteins or even the head proteins to better infect host cells. The effects of polymers on phage aggression are potentially highly productive. For example, if one knew, in general, how to use polymers to promote lytic phage aggression, one might be able to induce phage therapy via endogenous phages, excited to enter an aggressive state by a polymer embedded, for example, in a bandage (no exogenous phages added).

Polymers can be tested in cultures of two different types. First, a polymer is added to an aerated liquid culture. Second, the polymer is added to an upper layer, ultra-dilute agarose gel used for forming phage plaques. The same phage inoculum is added to a polymer-containing culture and a control without polymer. In the case of aerated liquid culture, polymer effectiveness is determined via the time until lysis. With some highly lytic phages (0305φ8-36, for example), a control, polymer-free culture will never lyse; the control cultures of other lytic phages may lyse, but relatively slowly. Thus, it is projected that lysis of the polymer-containing culture will occur more rapidly than the control culture in many cases. The time of lysis can be used as the measure of the effectiveness of the polymer type and concentration; the less the time of lysis is, the greater the effectiveness. This determination can be made at several polymer concentrations. The most effective polymer is the one that produces the lowest lysis time at any given polymer concentration.

For the upper layer gel cultures, speed of plaque diameter increase and the clarity of plaques can be used as the measures of the effectiveness of a given polymer type/concentration. The more rapidly growing and the more clear the plaque is, the greater is the effectiveness. All polymers will increase viscosity and, therefore, decrease thermal phage motion. Thermal phage motion promotes the finding and infecting of host cells. Thus, a polymer that is neutral in the stimulation of phage aggressiveness will, in fact, produce a decrease in the speed of phage propagation, via a decrease in thermal motion. Thus, non-specific, purely physical effects of the polymers are not a factor in producing a positive assay. That is to say, the assay understates the stimulation of lytic phage aggressiveness.

For cleared cultures, one can check the level of host cell destruction by either phase contrast or fluorescence microscopy. Turbidity can also decrease because the cells become smaller, even though still alive. Fluorescence microscopy can be used to determine whether phage aggregation contributes to plaque turbidity.

### III. Testing Optimal Phage Cocktails

To complete the work on phage therapy, database entries are made for all of the above characterizations and are processed to (1) rapidly design a highly effective phage cocktail against each targeted cell(s) presented to the investigator and (2) rapidly change the design in response to either a genomic change in the originally targeted cells (phage resistance, for example) or a change in the species of cells targeted.

While rapidly designing initial phage cocktails and rapidly changing phage cocktail design, database entries from 10's of thousands of lytic phages will be obtained. These data are entered, stored, maintained, and managed by a computer database that provides: (1) a list of phages that infect any given host, (2) optimal propagation conditions for each phage (especially any polymer stimulation observed), and (3) classification of each phage, which begins with physical size/genome length and ends with sequence annotation. The database will provide the information for mixing lytic phages of different type in amounts such that they do not, either as single phages, or in aggregate, initially infect at multiplicity high enough so that interference among them (superinfection immunity) inhibits effectiveness; avoidance of superinfection immunity limits the number of different phages in the cocktail. Superinfection immunity is a well-known, general phenomenon. Although these steps of cocktail design can be visually executed from the database, a computer-implemented method can be developed for this purpose.

Before use in phage therapy or prophylaxis, two tests are made of any phage cocktail: (a) A dilution series of the cocktail is performed and the maximum dilution (D_{M}-a) is determined for clearing a bacterial lawn after plating on a lawn grown in a growth medium containing, ultra-dilute agarose gel. The plate is incubated at the temperature of therapeutic use and for the time needed to form the average phage plaque, typically 10 hours at 30° C. This is done both with and without adding to the ultra-dilute agarose any polymer additives present in the cocktail. The effect of compounds likely to be present during therapeutic use is tested, keratin for example. The test with polymer additives determines the extent to which polymers are to be added to get full effectiveness for a local infection. The lawn-forming host bacterium is chosen to be the pathogen that is the target of the phage cocktail.

(b) One dilutes and plates as in (a), but determine the maximum dilution (D_{M}-b) for which a totally clear plate, without phage-resistant colonies, is produced and maintained for a longer time, typically between 48-96 hours, during continued incubation. Maximum dilution can gauge the long-range effectiveness of the cocktail in preventing emergence of bacterial resistance to the phages. Phage-resistance inevitably occurs when a single phage is used. Thus, dilution is important, especially since the primary (empirically established) reason for a cocktail (rather than a single phage) is to make the probability of phage resistance essentially zero.

The most effective cocktails are assumed to be those with the greatest values of D_{M}-a and D_{M}-b.

In addition, experience indicates that long-genome, lytic phages are expected that have not been accessible to past phage isolation by anyone else. To steer isolations in this high-potential direction, the initial focus is on isolating phages that make small (<1 mm) plaques in a 0.2% agarose overlay-contained lawn of host cells. These phages have been large dsDNA phages (genome length>200 Kb), as rapidly (hours) confirmed via (1) a relatively steep plaque radius vs. agarose gel concentration plot and then (2) relatively bright single phage appearance during in situ fluorescence microscopy. It is currently projected that optimized cocktails will have several large phages. As we learn more about the plaque morphologies of the most effective phages, this knowledge will be integrated into screens at the early stages of phage isolation. Unlike previous phage therapy isolates, phages compositions will be from dry, polymer-rich samples. Therefore, we will test use of phage aggressiveness-promoting polymers to also dry phage cocktails while maintaining phage activity; polymers dramatically stabilize phages.

Certain embodiments will be directed to compositions comprising phage cocktails that can be used for phage therapy. These cocktails will have been tested for both effectiveness and composition. They will be immediately useful for the phage therapy of animals.

To enhance the transportability and ease of delivering phage cocktails, phage cocktails will be transformed into dried cocktails. Dried cocktails are more transportable, storable at ambient temperature, and field-friendly. For example, dried cocktails can include embedding phages in (phage aggression-enhancing) polymer films that can be diced and mixed with animal feed for decreasing Salmonella enterica strains, many of them already multi-drug-resistant, in both cattle and pigs. Perhaps, even methanotrophs can be targeted in cattle, for the purposes of reducing global warming. The specificity of phages is to be considered for such an application in cattle, given that rumen microbial ecology is critical to health.

### IV. Pathogenic Bacteria

Administration of bacteriophage compositions capable of lysing, inhibiting the growth of, or disrupting biofilm formation of pathogenic bacteria to subjects is capable of improving the health of the subjects after administration. In other aspects the bacteriophage compositions can be used to reduce the contamination of various surfaces or locations. In particular, the methods provided herein are capable of improving the gastrointestinal health of the subjects. This may include reducing the incidence or severity of necrotic enteritis (by at least 10%, 15% or even 20% as compared to controls), or reducing the bacterial load in the intestines of the animal, specifically with regards to levels of at least one pathogenic bacteria. As used herein, pathogenic bacteria include bacteria capable of causing disease in the subjects or in a human. Disease includes mortality, morbidity, or reduced productivity of agricultural animals, e.g., reduced weight gain, reduced offspring, egg or milk production, or reduced feed conversion ratio. For example the levels of Salmonella, Campylobacter, E. coli or Clostridium perfringens in the gastrointestinal tract of animals may be reduced (at least 50% decrease in recovery, suitably at least 60%, 70%, 80% or even 90% decrease). Improving the gastrointestinal health may also be quantified by an increase in the daily average weight gain of an animal (at least 3% increase, suitably at least a 5%, 7%, 10%, 20%, 30%, 40% or even 50% increase in weight gain as compared to controls over a set period of time such as a week or month). A suitable control is a similar subject not administered a bacteriophage composition or the subject prior to administration of the bacteriophage composition.

The methods may also reduce the level or number of potential bacterial food-borne pathogens of humans in the gastrointestinal tract of commercial agricultural animals (at least 50% decrease in recovery, suitably at least 60%, 70%, 80% or even 90% decrease in recovery as compared to controls). In particular, the level of Salmonella and Campylobacter spp. in the gastrointestinal tract of animals may be reduced in animals administered a bacteriophage composition described herein.

Such a reduction in potential human pathogen load in the gastrointestinal tract of animals will limit the opportunity of contaminating the human food chain either during preparation of meat for human consumption or via contamination of animal products such as poultry eggs. In addition, reduction of the pathogenic bacterial load in animals treated with, the methods described herein may also reduce horizontal transmission of pathogenic bacteria within a group of animals (suitably horizontal transmission is reduced by at least 10%, 20%, 30%, 40% or as much as 50%). As used herein, pathogenic bacteria include any bacteria capable of causing morbidity or mortality in the animal being treated using the methods described herein or in immunocompetent humans.

Suitably the subjects used in the methods are humans, mammals or poultry, suitably the animals are domesticated agricultural animals such as cows, pigs, sheep, or poultry, suitably a chicken or turkey. If supplied in an animal feed, the feed may comprise between 10⁴, 10⁵, 10⁶, 10⁷, 10⁸, 10⁹, 10¹⁰, 10¹⁵ to 10¹⁰, 10¹¹, 10¹², 10¹³, 10¹⁴, 10¹⁵, 10¹⁶, 10^{17,} 10¹⁸, 10¹⁹, 10²⁰ bacteriophage/gm, including all values and ranges therein, of finished feed. The bacteriophage compositions may be added to feed during production, after production by the supplier or by the person feeding the animals, just prior to providing the feed to the animals. The bacteriophage may be provided as a single dosage form, administered simultaneously, or administered sequentially or completely separately.

In one embodiment a bacteriophage composition is used to neutralize and/or kill both active and stationary phase pathogenic bacteria. Examples of pathogenic bacteria include, but are not limited to, Escherichia, Salmonella, Listeria, Campylobacter, Shigella, Brucella, Helicobactor, Mycobacterium, Streptococcus, Staphylococcus, and Pseudomonas. In certain aspects the pathogenic bacteria is one or more of Bacillus (Bacillus anthracis), Bordetella (Bordetella pertussis), Borrelia (Borrelia burgdorferi), Brucella (Brucella abortus, Brucella canis, Brucella melitensis, Brucella suis), Campylobacter (Campylobacter jejuni), Chlamydia and Chlamydophila (Chlamydia pneumonia, Chlamydia trachomatis, Chlamydophila psittaci), Clostridium (Clostridium botulinum, Clostridium difficile, Clostridium perfringens, Clostridium tetani), Corynebacterium (Corynebacterium diphtheria), Enterococcus (Enterococcus faecalis, Enterococcus faecium), Escherichia (Escherichia coli), Francisella (Francisella tularensis), Haemophilus (Haemophilus influenza), Helicobacter (Helicobacter pylori), Legionella (Legionella pneumophila), Leptospira (Leptospira interrogans), Listeria (Listeria monocytogenes), Mycobacterium (Mycobacterium leprae, Mycobacterium tuberculosis, Mycobacterium ulcerans), Mycoplasma (Mycoplasma pneumonia), Neisseria (Neisseria gonorrhoeae, Neisseria meningitides), Pseudomonas (Pseudomonas aeruginosa), Rickettsia (Rickettsia rickettsii), Salmonella (Salmonella typhi, Salmonella typhimurium), Shigella (Shigella sonnei), Staphylococcus (Staphylococcus aureus, Staphylococcus epidermidis, Staphylococcus saprophyticus), Streptococcus (Streptococcus agalactiae, Streptococcus pneumonia, Streptococcus pyogenes), Treponema pallidum, Vibrio (Vibrio cholera), and Yersinia (Yersinia pestis). Examples of antibiotic resistant pathogenic bacteria include: various strains of Staphylococcus aureus (e.g., methicillin-resistant Staphylococcus aureus; MRSA), Streptococcus pyogenes, Enterococcus faecium, Pseudomonas aeruginosa, Clostridium difficile, E. coli, Salmonella, and Acinetobacter baumannii. Examples of Gram negative pathogenic bacteria include, but are not limited to Acinetobacter calcoaceficus, Aeromonas hydrophile, Enterobacter aerogenes, Escherichia coli ML-35, Escherichia coli O157:H7, Pseudomonas putida, Pseudomonas spp, Proteus mirabilis, Providencia stuartii, Salmonella, Salmonella Michigan, Salmonella Gaminola, Salmonella Montbidea, Salmonella Poona, Vibrio 01, Vibrio vulnificus CMCP6, Vibrio vulnificus M06, Vibrio sp., Vibrio parahaemolyticus P5, and the like.

### V. Bacteriophage

In certain embodiments 1, 2, 3, 4, 5, or more bacteriophage are isolated that are lytic for a targeted pathogenic bacterium or a number of pathogenic bacteria. A bacteriophage (phage) is a virus that infects and replicates within bacteria. Bacteriophages are composed of proteins that encapsulate a DNA or RNA genome, and may have relatively simple or elaborate structures. Their genomes may encode as few as four genes, and as many as hundreds of genes. Bacteriophage replicate within bacteria following the injection of their genome into the bacterial cytoplasm.

Bacteriophages are widely distributed in locations populated by bacterial hosts, locations that include soil and the intestines of animals. Even sea water, has up to 9×10⁸ virions per milliliter in microbial mats at the surface (Wommack and Colwell, Microbiology and Molecular Biology Reviews 64 (1): 69-114, 2000). Biofilms can have at least one million times more bacteriophage per volume than sea water, based on electron microscopy of thin sections. Thus, in certain aspects biofilms can be used as a source for bacteriophage. Bacteriophage have been used as an alternative to antibiotics and are seen as a possible therapy against multi-drug-resistant strains of bacteria.

The dsDNA tailed bacteriophages, or Caudovirales, account for 95% of bacteriophages reported in the scientific literature. Other bacteriophages occur in the biosphere, with different protein components (capsids), genomes, and lifestyles. Bacteriophages are classified according to morphology and nucleic acid by the International Committee on Taxonomy of Viruses (ICTV). Currently there are at least nineteen families of bacteriophage recognized. Of these, only two families have RNA genomes and only five families are enveloped by a membrane. Of the viral families with DNA genomes, only two have single-stranded genomes. Eight of the viral families with DNA genomes have circular genomes, while nine have linear genomes. Nine families infect bacteria only, nine infect archaea only, and one (Tectiviridae) infects both bacteria and archaea. Families of bacteriophage include Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttavirus, Inoviridae, Leviviridae, Microviridae, Plasmaviridae, and Tectiviridae. One or more bacteriophage can be selected for use in the methods described herein.

Current data indicate that roughly 10³¹ bacteriophages exist worldwide, including about 10⁸ genotypes and possibly most of the earth's gene diversity as estimated by metagenomics and fluorescence and electron microscopy (Breitbart and Rohwer Trends Microbiol 13:278-84, 2005; Brüssow and Kutter Phage ecology. In Bacteriophages: Biology and Applications Edited by: Kutter and Sulakvelidze, Boca Raton, Fla.: CRC Press; paes 129-63, 2005; Rohwer, Cell 113:141, 2003; Williamson et al., Appl Environ Microbiol 71:3119-25, 2005). Less than 1% of the observed bacteriophages have ever been grown in culture (sometimes called “the great plaque count anomaly”). The great plaque count anomaly is especially dramatic in the case of soil-borne bacteriophages. Propagated bacteriophages are sometimes not obtained from soil samples in spite of bacteriophage concentrations in the 10⁸-10⁹ range per gram, when detected by microscopy (Ashelford et al., Appl Environ Microbiol 69:285-89, 2003). Some bacteriophages, though viable, are probably not detected by any past procedures. Genomes of currently unpropagated bacteriophages are potentially a major source of unexplored environmental gene diversity.

Knowledge of environmental virus gene diversity has been recently expanded by sequencing of large eukaryotic phycodnaviruses and related viruses. These viruses have double-stranded DNA genomes with a length between 200 and 1,200 Kb (Claverie et al., Virus Res 117:133-44, 2006; Dunigan et al., Virus Res 117:119-32, 2006; Ghedin and Fraser, Trends Microbiol 13:56-57, 2005; Iyer et al., Virus Res 117:156-84, 2006). Large double-stranded DNA bacteriophages also exist, including Bacillus megaterium bacteriophage G (˜670 Kb genome (Hutson et al., Biopolymers 35:297-306, 1995)), Pseudomonas aeruginosa bacteriophage φKZ (280 Kb genome (Mesyanzhinov et al., J Mol Biol 317:1-19, 2002)) and several bacteriophages that are relatives of bacteriophage T4 by the criteria of DNA replication/recombination strategy, structure and interface of DNA replication to DNA packaging (Petrov et al., J Mol Biol 361:46-68, 2006; Nolan et al., Virol J3:30, 2006).

However, of the 5,400 or so bacteriophages that have been isolated (Ackermann, Classification of bacteriophages. In The bacteriophages Edited by: Calendar R. Oxford: Oxford University Press 8-16, 2006), 96% have double-stranded DNA genomes and of 405 deposited in databases, only 6 have genomes as long as 200 Kb. Two other T4-like bacteriophage genomes in draft status are also in this range (Petrov et al., J Mol Biol 361:46-68, 2006). Statistical analysis reveals a significant undersampling of long-genome bacteriophages (Claverie et al., Virus Res 117:133-44, 2006). The strong possibility exists that long-genome bacteriophages (>200 Kb genome) are more frequent and are major contributors to microbial ecology, but are under-sampled because of the use of classical bacteriophage propagation procedures and possibly also classical processing of environmental samples for microscopy. For example, bacteriophage G was discovered by accident ˜40 years ago through electron microscopy of a preparation of another bacteriophage (Donelli, Atti Accad Naz Lincei-Rend Clas Sci Fis Mat Nat 44:95-97, 1968). Long-genome bacteriophages are of interest for use in host/bacteriophage co-evolution.

To identify long-genome environmental bacteriophages, extraction and propagation can be performed in comparatively dilute agarose gels (e.g., 0.15% agarose gels). In certain aspects gels can contain nutrients or nutrient medium, such as 10 g Bacto tryptone, 5 g KCl in 1000 ml water with 0.002 M CaCl₂ (Serwer et al., Virology 329:412-24, 2004). Bacteriophages can be screened using single plaque cloning and determining the change in plaque size with change in supporting agarose gel concentration. For example, Bacillus thuringiensis bacteriophage 0305φ8-36 made small (<1 mm) plaques in a 0.4% agarose supporting gel. Plaques became progressively larger as the agarose gel concentration decreased to 0.2% and 0.15%. This dependence is comparatively steep, as confirmed in a side-by-side comparison with bacteriophages T4 and G. Post-isolation, 0305φ8-36 grew only in gels of either 0.25% or more dilute agarose (Serwer et al., Virol J (2007), 4:21).

1. A method of treating a pathogenic bacteria infection comprising:
(a) isolating two or more bacteriophage that lyse the pathogenic bacteria by
(i) placing an environmental sample on the surface of a solid nutrient broth growth medium;
(ii) applying ultra dilute (0.05%-0.02%) molten agarose containing the pathogenic bacteria and a nutrient broth onto the surface of the solid growth medium to completely or partially cover the environmental sample;
(iii) incubating agarose covered growth medium the pathogenic bacteria form a bacterial lawn in the ultra-dilute agarose layer and bacteriophage in the environmental sample form plaques in the pathogenic bacterial lawn;
(iv) sampling a plaque present in the ultradilute molten agarose;
(v) transferring the bacteriophage in the sampled plaque to a second agar growth medium;
(vi) applying a second ultra dilute (0.05%-0.02%) molten agarose containing the pathogenic bacteria and a nutrient broth to the second solid growth medium;
(ix) incubating the ultradilute agarose and second agar growth medium until the pathogenic bacteria formed a bacterial lawn in the ultra-dilute agarose layer and the transferred bacteriophage form plaques in the pathogenic bacterial lawn; and
(x) isolating the bacteriophage from the plaques from the second ultradilute agaraose; and
(b) formulating and administering two or more isolated bacteriophage that target the pathogenic bacteria infecting the subject.

2. The method of claim 1, further comprising rapid characterization of isolated bacteriophage by fluorescence microscopy.

3. The method of claim 1, wherein the isolated bacteriophage is characterized and a characterization profile is entered in database.

4. A phage composition comprising (a) long genome, lytic phage, and (b) lysis enhancing polymer.

5. A dry phage composition comprising at least two long-genome lytic phages that specifically bind and lyse a pathogenic bacterium.

6. The composition of claim 5, wherein the pathogenic bacterium is Salmonella enterica, Listeria monocytogenes, Escherichia coli (0157:H7), Campylobacter jejuni, Staphylococcus aureus and Clostridium perfringens.

7. Treating a subject having a bacterial infection comprising administering a long-genome, lytic phage composition comprising at least two phage that bind and lyse a pathogenic bacterium.
